(19) United States
(12) Patent Application Publication
(10) Pub. No.: US 2004/0170677 A1
(43) Pub. Date: Sep. 2, 2004

(54) Method of drug loading in liposomes by gradient

(76) Inventors: Ning Hu, San Gabriel, CA (US); Gerard M. Jensen, Brea, CA (US); Michele Sulivan, Arcadia, CA (US); Stephanie Yang, Temple City, CA (US)

Correspondence Address: Schwegman, Lundberg, Woessner & Kluth, P.A., P.O. Box 2938, Minneapolis, MN, 55402, US

(21) Appl. No.: 10/723,431

(22) Filed: Nov. 26, 2003

Related U.S. Application Data
(60) Provisional application No. 60/429,122, filed on Nov. 26, 2002.

(51) Int. Cl. A61K 9/127

(52) U.S. Cl. 424/450

(57) ABSTRACT

A method for encapsulation of pharmaceutical agents (e.g., antineoplastic agents) in liposomes is provided, having preferably a high drug:lipid ratio. Liposomes can be made by a process that loads the drug by an active mechanism using a transmembrane pH gradient. Using this technique, trapping efficiencies approach 100%. Drug:lipid ratios employed are higher than for older traditional liposome preparations, and the release rate of the drug from the liposomes is reduced. After loading, residual acid is quenched with a quenching agent that is base permeable at low temperatures. The residual aciditiy is thus reduced and chemical stability (e.g. against hydrolysis) is enhanced. The stability of both the liposome and the pharmaceutical agent is thus maintained, prior to administration. The pH gradient is, however, present when the liposome is administered in vivo because the quenching agent rapidly exits the liposome.

### PRIORITY OF INVENTION

This application claims priority from U.S. Provisional Application No. 60/429,122, filed 26 Nov. 2002.

### BACKGROUND OF THE INVENTION

Liposomes are completely closed lipid bilayer membranes containing an entrapped aqueous volume. Liposomes may be unilamellar vesicles (possessing a single membrane bilayer) or multilameller vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

The original liposome preparation of Bangham et al. (J. Mol. Biol., 1965, 13:238-252) involves suspending phospholipids in an organic solvent which is then evaporated to dryness leaving a phospholipid film on the reaction vessel. Next, an appropriate amount of aqueous phase is added, the mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys, Acta., 1967, 135:624-638), and large unilamellar vesicles.

Techniques for producing large unilamellar vesicles (LUVs), such as, reverse phase evaporation, infusion procedures, and detergent dilution, can be used to produce liposomes. A review of these and other methods for producing liposomes may be found in the text Liposomes, Marc Ostro, ed., Marcel Dekker, Inc., New York, 1983, Chapter 1. See also Szoka Jr. et al., (1980, Ann. Rev. Biophys. Bioeng., 9:467). A particularly preferred method for forming LUVs is described in Cullis et al., PCT Publication No. 87/00238, Jan. 16, 1986, entitled “Extrusion Technique for Producing Unilamellar Vesicles”.

Other techniques that are used to prepare vesicles include those that form reverse-phase evaporation vesicles (REV), Papahadjopoulos et al., U.S. Pat. No. 4,235,871. Another class of liposomes that can be used are those characterized as having substantially equal lamellar solute distribution. This class of liposomes is denominated as stable plurilamellar vesicles (SPLV) as defined in U.S. Pat. No. 4,522,803 to Lenk, et al. and includes monophasic vesicles as described in U.S. Pat. No. 4,588,578 to Fountain, et al. and frozen and thawed multilamellar vesicles (FATMLV) as described above.

In a liposome-drug delivery system, a bioactive agent such as a drug is entrapped in the liposome and then administered to the patient to be treated. For example, see Rahman et al., U.S. Pat. No. 3,993,754; Sears, U.S. Pat. No. 4,145,410; Paphadjopoulos et al., U.S. Pat. No. 4,235,871; Schneider, U.S. Pat. No. 4,224,179; Lenk et al., U.S. Pat. No. 4,522,803; and Fountain et al., U.S. Pat. No. 4,588,578. Alternatively, if the bioactive agent is lipophilic, it may associate with the lipid bilayer. Typically, the term “entrapment” includes both the drug in the aqueous volume of the liposome as well as drug associated with the lipid bilayer.

Doxorubicin is a widely used antineoplastic drug belonging to the anthracycline class of antibiotics produced by the fungi, Streptomyces peucetius. Doxorubicin has been utilized against a variety of tumors, leukemias, sarcomas, and breast cancer. Toxicities seen with commonly administered doses of doxorubicin (as well as other antineoplastic agents) include myelosuppression, alopecia, mucositis, and gastrointestinal toxicities including nausea, vomiting, and anorexia. The most serious doxorubicin toxicity is cumulative dose-dependent irreversible cardiomyopathy leading to congestive heart failure in 1-10 percent of patients receiving doses greater than 550 mg per square meter of body area. These toxicities severely limit the clinical utility of antineoplastic agents such as doxorubicin.

As has been established by various investigators, cancer therapy employing antineoplastic agents can in many cases be significantly improved by encapsulating the antineoplastic agent in liposomes using traditional methods, rather than administering the free agent directly into the body. See, for example, Forssen, et al., (1983), Cancer Res., 43:546; and Gabizon et al., (1982), Cancer Res., 42:4734. Passive incorporation of such agents in liposomes can change their antitumor activities, clearance rates, tissue distributions, and toxicities compared to direct administration. See, for example, Rahman et. al., (1982), Cancer Res., 42:1817; Rosa, et al., (1982) in Transport in Biomembranes: Model Systems and Reconstitution, R. Antoline et al., ed. Raven Press, New York. 243-256; Rosa, et al., (1983), Pharmacology, 26:221; Gabizon et al., (1983), Cancer Res., 43:4730; Forssen et al., supra; Gabizon, et al., supra; and Olson, et al., (1982), Br. J. Cancer Clin. Oncol., 18:167. Utilizing liposomes of various composition and size, evidence has been gathered demonstrating that the acute and chronic toxicities of doxorubicin can be attenuated by directing the drug away from target organs. For example, it is known that the cardiotoxicity of the anthracycline antibiotics daunorubicin and doxorubicin and their pharmaceutically acceptable derivatives and salts can be significantly reduced through passive liposome encapsulation. See, for example, Forssen-et al., supra; Olson et al., supra; and Rahman et al., supra. This buffering of toxicity appears mainly to arise from reduced accumulation into the heart, with associated reduction in cardiotoxicity (Rahman et al., 1980 Cancer Res., 40:1532; Olson et al., supra.; Berman et al., 1983, Cancer Res., 43:5427; and Rahman et al., 1985, Cancer Res., 45:796). Such toxicity is normally cumulative dose limiting for free doxorubicin (Minow et al., 1975, Cancer Chemother. Rep. 6:195). Incorporation of highly toxic antineoplastic agents in liposomes can also reduce the risk of exposure to such agents by persons involved in their administration.

Although the above-mentioned studies clearly established the potential for use of liposomally encapsulated antineoplastic agents such as doxorubicin, a commercially acceptable liposomal preparation has not been available from the types of liposomes described above. For example, many of these formulations have dubious pharmaceutical potential due to problems associated with stability, trapping efficiency, scaleup potential, and cost of the lipids used. In addition, problems related to the efficiency with which drugs are encapsulated have been encountered. Such problems have accompanied the passive entrapment methods used heretofore.

Yet another problem with prior antineoplastic agent-containing liposomes is that none of the previous liposomal formulations of antineoplastic agent fully satisfy fundamental stability demands. Retention of antineoplastic agent within a liposomal preparation is commonly measured in hours, whereas pharmaceutical applications commonly require stabilities of a year or more. Further, the chemical stability of component lipids is questionable due to the high proportion of very unsaturated lipids such as cardiolipin. Other problems include the high cost of negatively charged lipids and scale-up problems. Due to the fact that antineoplastic agents such as doxorubicin have an amphipathic nature, it is permeable to bilayer membranes rendering the liposome preparations unstable due to leakage of the drug from the vesicles (Gabizon et al., 1982, supra.; Rahman et al., 1985, supra; and Ganapathi et al., 1984, Biochem. Pharmacol., 33:698).

Mayer et al. found that the problems associated with efficient liposomal entrapment of the antineoplastic agent can be alleviated by employing transmembrane ion gradients (see PCT application 86/01102, published Feb. 27, 1986). Aside from inducing doxorubicin uptake, such transmembrane gradients also act to increase drug retention in the liposomes.

Liposomes themselves have been reported to have no significant toxicities in previous human clinical trials where they have been given intravenously. Richardson et al., (1979), Br. J. Cancer 40:35; Ryman et al., (1983) in “Targeting of Drugs” G. Gregoriadis, et al., eds. pp 235-248, Plenum, N.Y.; Gregoriadis G., (1981), Lancet 2:241, and Lopez-Berestein et al., (1985) J. Infect. Dis., 151:704. Liposomes are reported to concentrate predominantly in the reticuloendothelial organs lined by sinosoidal capillaries, i.e., liver, spleen, and bone marrow, and phagocytosed by the phagocytic cells present in these organs.

The use of liposomes to administer antineoplastic agents has raised problems with regard to both drug encapsulation and trapping efficiencies, and drug release during therapy. With regard to encapsulation, there has been a continuing need to increase trapping efficiencies so as to minimize the lipid load presented to the patient during therapy. In addition, high trapping efficiencies mean that only a small amount of drug is lost during the encapsulation process, an important advantage when dealing with the expensive drugs currently being used in cancer therapy. As to drug release, many antineoplastic agents, such as doxorubicin, have been found to be rapidly released from traditional liposomes after encapsulation. Such rapid release diminishes the beneficial effects of liposome encapsulation on efficacy and accelerates release of the drug into the circulation, causing toxicity, and thus, in general, is undesirable. Accordingly, there have been continuing efforts by workers in the art to find ways to reduce the rate of release of antineoplastic agents and other drugs from liposomes.

In addition to these problems with encapsulation and release, there is the overriding problem of finding a commercially acceptable way of providing liposomes containing antineoplastic agents to the clinician. Although the production and loading of liposomes on an “as needed” basis is an acceptable procedure in an experimental setting, it is generally unsatisfactory in a clinical setting. Accordingly, there is a significant and continuing need for methods whereby liposomes, with or without encapsulated drugs, can be shipped, stored and in general moved through conventional commercial distribution channels without substantial damage.

DaunoXome, with 50 mM citric acid gradient loaded daunorubicin, has been commercialized. Doxil, which is a liposomal doxorubicin with pegylated lipids, has also been commercialized but the doxorubicin drug is loaded against an ammonium sulfate ion gradient, rather than acid gradient loading.

Published PCT Patent Application WO 99/13816 to Moynihan et al. discloses liposomal camptothecin formulations and processes for making the same. The process includes hydrating a dehydrated liposome (film or powder) with an aqueous solution containing an excipient having a pH range from 2.0 to 7.4 to form a liposome dispersion. The preferred aqueous solution for purposes of hydration, disclosed therein, is a buffered solution of the acid, sodium of ammonium forms of citrate or sulfate. The preferred buffers disclosed therein are >5 mM, more preferably 50 mM, citric acid (pH 2.0-5.0), ammonium citrate (pH 2.0-5.5), or ammonium sulfate (pH 2.0 to 5.5). See, page 12, lines 12-23. Published PCT Patent Application WO 99/13816 also describes that once loaded, the liposomal formulation is quenched with ammonium sulfate.

Published PCT Patent Application WO 99/13816, however, does not teach or suggest that upon administration of the liposomal formulation, that the original gradient is attained. Additionally, the published PCT patent application does not teach or suggest that citric acid greater than 50 mM can be employed (although it does generically state above 5 nM), while maintaining the ability to load relatively large amounts of drug (GI147211, a camptothecin analog). The published PCT patent application does not teach or suggest that drugs other than camptothecin can be employed in such liposomal formulations.

### SUMMARY OF THE INVENTION

A method for encapsulation of pharmaceutical agents (e.g., antineoplastic agents) in liposomes is provided, having preferably a high drug:lipid ratio. Liposomes can be made by a process that loads the drug by an active mechanism using a transmembrane pH gradient. Using this technique, trapping efficiencies approach 100%. Drug:lipid ratios employed are higher than for older traditional liposome preparations, and the release rate of the drug from the liposomes is reduced. After loading, residual acid is quenched with a quenching agent that is base permeable at low temperatures. The residual aciditiy is thus reduced and chemical stability (e.g. against hydrolysis) is enhanced. The stability of both the liposome and the pharmaceutical agent is thus maintained, prior to administration. The pH gradient is, however, present when the liposome is administered in vivo because the quenching agent rapidly exits the liposome.

The present invention provides a method of forming gradient loaded liposomes having a lower inside/higher outside pH gradient. The method includes: (a) contacting a solution of liposomes with a pharmaceutical agent in an aqueous solution of at least about 60 mM of an acid, at a temperature wherein the protonated form of the pharmaceutical agent is charged and is not capable of permeating the membrane of the liposomes, and wherein the unprotonated form of the pharmaceutical agent is uncharged and is capable of permeating the membrane of the liposomes; (b) cooling the solution to a temperature at which the unprotonated form of the pharmaceutical agent is not capable of permeating the membrane of the liposomes; and (c) contacting the solution with a weak base, in an amount effective to raise the pH of the internal liposome to provide gradient loaded liposomes having a lower inside/higher outside pH gradient.

The present invention also provides a method for preparing a pharmaceutical composition. The method includes (a) contacting a solution of liposomes with a pharmaceutical agent in an aqueous solution of at least about 60 mM of an acid, at a temperature wherein the protonated form of the pharmaceutical agent is charged and is not capable of permeating the membrane of the liposomes, and wherein the unprotonated form of the pharmaceutical agent is uncharged and is capable of permeating the membrane of the liposomes; (b) cooling the solution to a temperature at which the unprotonated form of the pharmaceutical agent is not capable of permeating the membrane of the liposomes; (c) contacting the solution with a weak base, in an amount effective to raise the pH of the internal liposome to provide gradient loaded liposomes having a lower inside/higher outside pH gradient; and (d) combining the liposomes with a pharmaceutically acceptable carrier to provide the pharmaceutical composition.

The present invention also provides a method that includes administering the pharmaceutical composition of the present invention to a mammal.

The present invention also provides a method for treating a mammal inflicted with cancer. The method includes administering the pharmaceutical composition of the present invention to the mammal, wherein the pharmaceutical agent is an antineoplastic agent.

The present invention also provides a gradient loaded liposome having a lower inside/higher outside pH gradient, wherein the gradient loaded liposome is prepared by the process that includes: (a) contacting a solution of liposomes with a pharmaceutical agent in an aqueous solution of at least about 60 mM of an acid, at a temperature wherein the protonated form of the pharmaceutical agent is charged and is not capable of permeating the membrane of the liposomes, and wherein the unprotonated form of the pharmaceutical agent is uncharged and is capable of permeating the membrane of the liposomes; (b) cooling the solution to a temperature at which the unprotonated form of the pharmaceutical agent is not capable of permeating the membrane of the liposomes; and (c) contacting the solution with a weak base, in an amount effective to raise the pH of the internal liposome to provide gradient loaded liposomes having a lower inside/higher outside pH gradient.

### BRIEF DESCRIPTION OF THE DRAWINGS

Embodiments of the invention may be best understood by referring to the following description and accompanying drawings which illustrate such embodiments. In the drawings:

FIG. 1 illustrates the effect of liposomal vinorelbine on human breast tumor MaTu growth in mice.

FIG. 2 illustrates a block flow diagram for preparing liposomal formulations via methods of the present invention.

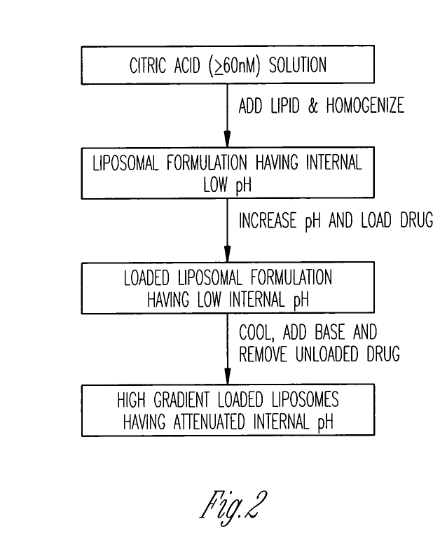

### DETAILED DESCRIPTION OF THE INVENTION

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention provides for an efficient trapping of antineoplastic agents in liposomes exhibiting a transmembrane pH gradient. The liposomal formulations of the present invention, upon administration, provide liposomes having substantially the original pH gradient. The liposomes of the present invention possess a drug to lipid ratio significantly higher than older traditional liposomal systems. The liposomal formulations of the present invention can be used as drug carrier systems that entrap drugs such as antineoplastic agents. The liposomes of the present invention have improved pharmacokinetics, enhanced efficacy (bioactivity), lower toxicity, and provide an improved therapeutic index as compared to the free drug. As such, when the liposomal formulations of the present invention are used as drug carrier systems that entrap toxic antineoplastic agents such as anthracyclines (e.g., doxorubicin, epirubicin, and daunorubicin); anthracenediones (e.g., mitoxantrone); vinca alkaloids (e.g., vincristine and vinblastine); antineoplastic antibiotics; an alkylating agent (e.g., cyclophosphamide and mechlorethamine hydrochloride); and purine or pyrimidine derivatives (e.g., 5-fluorouracil), such liposomal formulations can be used to decrease the toxic effects of the antineoplastic agent.

The present invention relates to novel methods of preparing liposomal formulations, to the liposomal formulations obtained from such processes, as well as methods of medical treatment that include administering the liposomal formulations. When describing the methods, products obtained from such methods, formulations that include such products, and methods of using such products, the following terms have the following meanings, unless otherwise indicated.

Definitions

As used herein, the term “liposome” refers to unilamellar vesicles or multilamellar vesicles such as are described in U.S. Pat. No. 4,753,788.

“Unilamellar liposomes,” also referred to as “single lamellar vesicles,” are spherical vesicles that includes one lipid bilayer membrane which defines a single closed aqueous compartment. The bilayer membrane includes two layers of lipids; an inner layer and an outer layer (leaflet). The outer layer of the lipid molecules are oriented with their hydrophilic head portions toward the external aqueous environment and their hydrophobic tails pointed downward toward the interior of the liposome. The inner layer of the lipid lays directly beneath the outer layer, the lipids are oriented with their heads facing the aqueous interior of the liposome and their tails toward the tails of the outer layer of lipid.

“Multilamellar liposomes” also referred to as “multilamellar vesicles” or “multiple lamellar vesicles,” include more than one lipid bilayer membrane, which membranes define more than one closed aqueous compartment. The membranes are concentrically arranged so that the different membranes are separated by aqueous compartments, much like an onion.

The term pharmaceutical agent includes but is not limited to, an analgesic, an anesthetic, an antiacne agent, an antibiotic, an antibacterial, an anticancer, an anticholinergic, an anticoagulant, an antidyskinetic, an antiemetic, an antifibrotic, an antifungal, an antiglaucoma agent, an anti-inflammatory, an antineoplastic, an antiosteoporotic, an antipagetic, an anti-Parkinson's agent, an antisporatic, an antipyretic, an antiseptic, an antithrombotic, an antiviral, a calcium regulator, a keratolytic, or a sclerosing agent.

The terms “encapsulation” and “entrapped,” as used herein, refer to the incorporation or association of the pharmaceutical agent in or with a liposome. The pharmaceutical agent may be associated with the lipid bilayer or present in the aqueous interior of the liposome, or both. In one embodiment, a portion of the encapsulated pharmaceutical agent takes the form of a precipitated salt in the interior of the liposome. The pharmaceutical agent may also self precipitate in the interior of the liposome.

The terms “excipient” “counterion” and “counterion excipient,” as used herein, refer to a substance that can initiate or facilitate drug loading and may also initiate or facilitate precipitation of the pharmaceutical agent in the aqueous interior of the liposome. Examples of excipients include, but are not limited to, the acid, sodium or ammonium forms of monovalent anions such as chloride, acetate, lactobionate and formate; divalent anions such as aspartate, succinate and sulfate; and trivalent ions such as citrate and phosphate. Preferred excipients include citrate and sulfate.

“Phospholipid” refers to any one phospholipid or combination of phospholipids capable of forming liposomes. Phosphatidylcholines (PC), including those obtained from egg, soy beans or other plant sources or those that are partially or wholly synthetic, or of variable lipid chain length and unsaturation are suitable for use in the present invention. Synthetic, semisynthetic and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC) and dimyristoylphosphatidylcholine (DMPC) are suitable phosphatidylcholines for use in this invention. All of these phospholipids are commercially available. Preferred PCs are HSPC and DSPC; the most preferred is HSPC.

Further, phosphatidylglycerols (PG) and phosphatic acid (PA) are also suitable phospholipids for use in the present invention and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is the preferred negatively charged lipid when used in formulations. Other suitable phospholipids include phosphatidylethanolamines phosphatidylinositols, and phosphatidic acids containing lauric, myristic, stearoyl, and palmitic acid chains. Further, incorporation of polyethylene glycol (PEG) containing phospholipids is also contemplated by the present invention.

The term “parenteral” as used herein refers to intravenous (IV), intramuscular (IM), subcutaneous (SubQ) or intraperitoneal (IP) administration.

The term “improved therapeutic index” refers to a higher therapeutic index relative to the free drug. The therapeutic index can be expressed as a ratio of the lethal dose for 50% of the animals relative to the effective dose.

As used herein, “treat” or “treating” refers to: (i) preventing a pathologic condition (e.g., breast cancer) from occurring (e.g. prophylaxis) or symptoms related to the same; (ii) inhibiting the pathologic condition or arresting its development or symptoms related to the same; or (iii) relieving the pathologic condition or symptoms related to the same.

It is contemplated by this invention to optionally include cholesterol in the liposomal formulation. Cholesterol is known to improve liposome stability and prevent loss of phospholipid to lipoproteins in vivo.

Any suitable lipid: pharmaceutical agent ratio that is efficacious is contemplated by this invention. Preferred lipid: pharmaceutical agent molar ratios include about 5:1 to about 100:1, more preferably about 10:1 to about 40:1. The most preferred lipid: pharmaceutical agent molar ratios include about 15:1 to about 25:1. Preferred liposomal formulations include phospholipid:cholesterol molar ratios over the range of 1.5:0.5 to 2:1.5. Most preferred liposomal formulation is 2:1 PC:chol with or without 1 to 4 mole percent of a phosphatidylglycerol. The most preferred liposomal size is less than 100 nm. The preferred loading efficiency of pharmaceutical agent is a percent encapsulated pharmaceutical agent of about 70% or greater. Encapsulation includes molecules present in the interior aqueous space of the liposome, molecules in the inner or outer leaflet of the membrane bilayer, molecules partially buried in the outer leaflet of the bilayer and partially external to the liposome, and molecules associated with the surface of the liposome, e.g., by electrostatic interactions.

Generally, the process of preparing the formulation embodied in the present invention is initiated with the preparation of a solution from which the liposomes are formed. This is done, for example, by weighing out a quantity of a phosphatidylcholine optionally cholesterol and optionally a phosphatidylglycerol and dissolving them in an organic solvent, preferably chloroform and methanol in a 1:1 mixture (v/v) or alternatively neat chloroform. The solution is evaporated to form a solid lipid phase such as a film or a powder, for example, with a rotary evaporator, spray dryer or other means. The film or powder is then hydrated with an aqueous solution containing an excipient having a pH range from 2.0 to 7.4 to form a liposome dispersion. The preferred aqueous solution for purposes of hydration is a buffered solution of the acid, sodium or ammonium forms of citrate or sulfate. The preferred buffers are at least about 60 mM, citric acid (pH 2.0-5.0), ammonium citrate (pH 2.0-5.5), or ammonium sulfate (pH 2.0 to 5.5). It would be known by one of skill in the art that other anionic acid buffers could be used, such as phosphoric acid. The lipid film or powder dispersed in buffer is heated to a temperature from about 25° C. to about 70° C. depending on the phospholipids used.

The liposomes formed by the procedure of the present invention can be lyophilized or dehydrated in the presence of a hydrophilic agent.

Multilamellar liposomes are formed by agitation of the dispersion, preferably through the use of a thin-film evaporator apparatus such as is described in U.S. Pat. No. 4,935,171 or through shaking or vortex mixing. Unilamellar vesicles are formed by the application of a shearing force to an aqueous dispersion of the lipid solid phase, e.g., by sonication or the use of a microfluidizing apparatus such as a homogenizer or a French press. Shearing force can also be applied using either injection, freezing and thawing, dialyzing away a detergent solution from lipids, or other known methods used to prepare liposomes. The size of the liposomes can be controlled using a variety of known techniques including the duration of shearing force. Preferably, a homogenizing apparatus is employed to from unilamellar vesicles having diameters of less than 200 nanometers at a pressure of 3,000 to 14,000 psi preferably 10,000 to 14,000 psi, and a temperature of about the aggregate transition temperature of the lipids.

Unentrapped excipient may or may not be removed or exchanged from the liposome dispersion by buffer exchange to 9% sucrose using either dialysis, size exclusion column chromatography (Sephadex G-50 resin) or ultrafiltration (100,000-300,000 molecular weight cut off). Each preparation of small unilamellar liposomes is then actively loaded with drug, for approximately 10-30 minutes against a gradient, such as a membrane potential, generated as the external pH is titrated to the range of 5.0 or above with sodium hydroxide. The temperature ranges during the drug loading step is generally between about 50° C.-70° C. with lipid:drug ratios between 5:1 to 100:1. Unentrapped pharmaceutical agent is removed from the liposome dispersion by buffer exchange to 9% sucrose using either dialysis, size exclusion column chromatography (Sephadex G-50 resin) or ultrafiltration (100,000-300,000 molecular weight cut off). Samples are generally filtered at about 55° C.-65° C. through a 0.22 micron filter composed of either cellulose acetate or polyether sulfone.

As described above, the pharmaceutical agent is generally loaded into preformed liposomes using known loading procedures (see for example Deamer et al. BBA 274:323-335 (1972); Forssen U.S. Pat. No. 4,946,683; Cramer et al. BBRC 75:295-301 (1977); Bally U.S. Pat. No. 5,077,056). The loading is by pH gradient. It is preferable to begin with an internal pH of approximately pH 2-3. The excipient is the counterion in the loading process and when it comes in contact with the pharmaceutical agent in the interior of the liposome, the excipient may cause a substantial portion of the pharmaceutical agent to precipitate. The pharmaceutical agent may also self precipitate in the interior of the liposome. This precipitation protects the pharmaceutical agent and the lipids from degradation (e.g., hydrolysis). An excipient, such as citrate or sulfate, may precipitate the pharmaceutical agent and can be utilized in the interior of the liposomes together with a gradient (pH or ammonia) to promote drug loading.

Drug loading via the pH gradient includes a low pH in the internal aqueous space of the liposomes, and this internal acidity is, by design, incompletely neutralized during the drug loading process. This residual internal acidity can cause chemical instability in the liposomal preparation (e.g., lipid hydrolysis), leading to limitations in shelf life. To quench this residual internal acidity, membrane permeable bases, such as amines (e.g., ammonium salts or alkyl-amines) can be added following the loading of the pharmaceutical agent in an amount sufficient to reduce the residual internal acidity to a minimum value (for example, pH at or above 4). Ammonium salts that can be used include ones having mono- or multi-valent counterions, such as, but not limited to, ammonium sulfate, ammonium hydroxide ammonium acetate, ammonium chloride, ammonium phosphate, ammonium citrate, ammonium succinate, ammonium lactobionate, ammonium carbonate, ammonium tartrate, and ammonium oxalate. The analogous salt of any alkyl-amine compound which is membrane permeable can also be used, including, but not limited to, methylamine, ethylamine, diethylamine, ethylenediamine, and propylamine. During storage, for example at 2-8C, the liposomal preparation will remain quenched, with reduced propensity for hydrolysis of either excipients or drug, relative to an un-quenched formulation. Upon injection, however, this quenching species rapidly leaks out of the liposome, thus restoring the residual gradient, which gradient is necessary for drug retention in vivo.

The therapeutic use of liposomes can include the delivery of drugs which are normally toxic in the free form. In the liposomal form, the toxic drug may be directed away from the sensitive tissue where toxicity can result and targeted to selected areas where they can exert their therapeutic effects. Liposomes can also be used therapeutically to release drugs slowly, over a prolonged period of time, thereby reducing the frequency of drug administration through an enhanced pharmacokinetic profile. In addition, liposomes can provide a method for forming an aqueous dispersion of hydrophobic drugs for intravenous delivery.

The route of delivery of liposomes can also affect their distribution in the body. Passive delivery of liposomes involves the use of various routes of administration e.g., parenterally, although other effective administration forms, such as intraarticular injection, inhalant mists, orally active formulations, transdermal iotophoresis or suppositories are also envisioned. Each route produces differences in localization of the liposomes.

The invention also provides a method of inhibiting the growth of tumors, both drug resistant and drug sensitive, by delivering a therapeutic or effective amount of liposomal camptothecin to a tumor, preferably in a mammal. Because dosage regimens for pharmaceutical agents are well known to medical practitioners, the amount of the liposomal pharmaceutical agent formulations which is effective or therapeutic for the treatment of the above mentioned diseases or conditions in mammals and particularly in humans will be apparent to those skilled in the art. The optimal quantity and spacing of individual dosages of the formulations herein will be determined by the nature and extent of the condition being treated, the form, route and site of administration, and the particular patient being treated, and such optimums can be determined by conventional techniques. It will also be appreciated by one of skill in the art that the optimal course of treatment, i.e., the number of doses given per day for a defined number of days, can be ascertained by those skilled in the art using conventional course of treatment determination tests

Inhibition of the growth of tumors associated with all cancers is contemplated by this invention, including multiple drug resistant cancer. Cancers for which the described liposomal formulations may be particularly useful in inhibiting are ovarian cancer, small cell lung cancer (SCLC), non small cell lung cancer (NSCLC), colorectal cancer, breast cancer, and head and neck cancer. In addition, it is contemplated that the formulations described and claimed herein can be used in combination with existing anticancer treatments. For example, the formulations described herein can be used in combination with taxanes such as (1) Taxol (paclitaxel) and platinum complexes for treating ovarian cancer; (2) 5FU and leucovorin or levamisole for treating colorectal cancer; and (3) cisplatin and etoposide for treating SCLC.

The liposomes containing therapeutic agents (e.g., antineoplastic agents) and the pharmaceutical formulations thereof of the present invention and those produced by the processes thereof can be used therapeutically in animals (including man) in the treatment of infections or conditions which require: (1) repeated administrations, (2) the sustained delivery of the drug in its bioactive form, or (3) the decreased toxicity with suitable efficacy compared with the free drug in question. Such conditions include but are not limited to neoplasms such as those that can be treated with antineoplastic agents.

The mode of administration of the liposomes containing the pharmaceutical agents (e.g., antineplastic agents) and the pharmaceutical formulations thereof determine the sites and cells in the organism to which the compound will be delivered. The liposomes of the present invention can be administered alone but will generally be administered in admixture with a pharmaceutical carrier selected with regard to the intended route of administration and standard pharmaceutical practice. The preparations may be injected parenterally, for example, intravenously. For parenteral administration, they can be used, for example, in the form of a sterile aqueous solution which may contain other solutes, for example, enough salts or glucose to make the solution isotonic. The doxorubicin liposomes, for example, may be given, as a 60 minute intravenous infusion at a dose of at least about 20 mg/m². They may also be employed for peritoneal lavage or intrathecal administration via injection. They may also be administered subcutaneously for example at the site of lymph node metastases. Other uses, depending on the particular properties of the preparation, may be envisioned by those skilled in the art.

For the oral mode of administration, the liposomal therapeutic drug (e.g., antineoplastic drug) formulations of this invention can be used in the form of tablets, capsules; losenges, troches, powders, syrups, elixirs, aqueous solutions and suspensions, and the like. In the case of tablets, carriers which can be used include lactose, sodium citrate and salts of phosphoric acid. Various disintegrants such as starch, and lubricating agents, such as magnesium stearate, sodium lauryl sulfate and talc, are commonly used in tablets. For oral administration in capsule form, useful diluents are lactose and high molecular weight polyethylene glycols. When aqueous suspensions are required for oral use, the active ingredient is combined with emulsifying and suspending agents. If desired, certain sweetening and/or flavoring agents can be added.

For the topical mode of administration, the liposomal therapeutic drug (e.g., antineoplastic drug) formulations of the present invention may be incorporated into dosage forms such as gels, oils, emulsions, and the like. Such preparations may be administered by direct application as a cream, paste, ointment, gel, lotion or the like.

For administration to humans in the curative, remissive, retardive, or prophylactic treatment of neoplastic diseases the prescribing physician will ultimately determine the appropriate dosage of the neoplastic drug for a given human subject, and this can be expected to vary according to the age, weight, and response of the individual as well as the nature and severity of the patient's disease. The dosage of the drug in liposomal form will generally be about that employed for the free drug. In some cases, however, it may be necessary to administer dosages outside these limits.

Specific ranges and values in the enumareated embodiments provided below are for illustration purposes only and do not otherwise limit the scope of the invention, as defined by the claims.

### Enumerated Embodiments of the Invention

[1] The present invention provides an improved method of forming gradient loaded liposomes having a lower inside/higher outside pH gradient, the method comprising:

(a) contacting a solution of liposomes with a pharmaceutical agent in an aqueous solution of at least about 60 mM of an acid, at a temperature wherein the protonated form of the pharmaceutical agent is charged and is not capable of permeating the membrane of the liposomes, and wherein the unprotonated form of the pharmaceutical agent is uncharged and is capable of permeating the membrane of the liposomes;

(b) cooling the solution to a temperature at which the unprotonated form of the pharmaceutical agent is not capable of permeating the membrane of the liposomes; and

(c) contacting the solution with a weak base, in an amount effective to raise the pH of the internal liposome to provide gradient loaded liposomes having a lower inside/higher outside pH gradient.

[2] The present invention also provides the method of embodiment [1], wherein the liposomes comprise phosphatidylcholine.

[3] The present invention also provides the method of any one of embodiments [1]-[2], wherein the liposomes comprise phosphatidylcholine selected from the group of distearoylphosphatidylcholine, hydrogenated soy phosphatidylcholine, hydrogenated egg phosphatidylcholine, dipalmitoylphosphatidylcholine, dimyristoylphosphatidylcholine, and dielaidoyl phosphatidyl chline.

[4] The present invention also provides the method of any one of embodiments [1]-[3], wherein the liposomes further comprise cholesterol.

[5] The present invention also provides the method of any one of embodiments [1]-[4], wherein the liposomes further comprise phosphatidylglycerol.

[6] The present invention also provides the method of any one of embodiments [1]-[5], wherein the liposomes further comprise non-phosphatidyl lipids.

[7] The present invention also provides the method of embodiment [6], wherein the non-phosphatidyl lipids comprise sphingomyelin.

[8] The present invention also provides the method of any one of embodiments [1]-[7], wherein the liposomes further comprise phosphatidylglycerol selected from the group of dimyristoylphosphatidylglycerol, dilaurylphosphatidylglycerol, dipalmitoylphosphatidylglycerol, and distearoylphosphatidylglycerol.

[9] The present invention also provides the method of any one of embodiments [1]-[8], wherein the liposomes comprises phosphatidylcholine, and further comprises cholesterol.

[10] The present invention also provides the method of any one of embodiments [1]-[9], wherein the liposomes comprises phosphatidylcholine, and further comprises cholesterol, wherein the molar ratio of the phosphatidylcholine to the cholesterol is about 1:0.01 to about 1:1.

[11] The present invention also provides the method of any one of embodiments [1]-[10], wherein the liposomes comprises phosphatidylcholine, and further comprises cholesterol, wherein the molar ratio of the phosphatidylcholine to the cholesterol is about 1.5:1.0 to about 3.0:1.0.

[12] The present invention also provides the method of any one of embodiments [1]-[11], wherein the liposomes are unilamellar and less than about 100 nm.

[13] The present invention also provides the method of any one of embodiments [1]-[12], wherein the weight ratio of the liposomes to the pharmaceutical agent is up to about 200:1.

[14] The present invention also provides the method of any one of embodiments [1]-[13], wherein the weight ratio of the liposomes to the pharmaceutical agent is about 1:1 to about 100:1.

[15] The present invention also provides the method of any one of embodiments [1]-[14], wherein the weight ratio of the liposomes to the pharmaceutical agent is about 1:1 to about 50:1.

[16] The present invention also provides the method of any one of embodiments [1]-[15], wherein the acid has an acid dissociation constant of less than about 1×10⁻².

[17] The present invention also provides the method of any one of embodiments [1]-[16], wherein the acid has an acid dissociation constant of less than about 1×10⁻⁴.

[18] The present invention also provides the method of any one of embodiments [1]-[17], wherein the acid has an acid dissociation constant of less than about 1×10⁻⁵.

[19] The present invention also provides the method of any one of embodiments [1]-[18], wherein the acid has a permeability coefficient larger than about 1×10⁻⁴ cm/sec for the liposomes.

[20] The present invention also provides the method of any one of embodiments [1]-[19], wherein the acid is selected from the group of formic acid, acetic acid, propanoic acid, butanoic acid, pentanoic acid, citric acid, oxalic acid, succinic acid, lactic acid, malic acid, tartaric acid, fumaric acid, benzoic acid, aconitic acid, veratric acid, phosphoric acid, sulfuric acid, and combinations thereof.

[21 ] The present invention also provides the method of any one of embodiments [1]-[20], wherein the acid is citric acid.

[22] The present invention also provides the method of any one of embodiments [1]-[21], wherein at least about 100 mM of an acid is employed.

[23] The present invention also provides the method of any one of embodiments [1]-[22], wherein the pharmaceutical agent exists in a charged state when dissolved in an aqueous medium.

[24] The present invention also provides the method of any one of embodiments [1]-[23], wherein the pharmaceutical agent is an organic compound that includes at least one acyclic or cyclic amino group, capable of being protonated.

[25] The present invention also provides the method of any one of embodiments [1]-[24], wherein the pharmaceutical agent is an organic compound that includes at least one primary amine group, at least one secondary amine group, at least one tertiary amine group, at least one quaternary amine group, or any combination thereof.

[26] The present invention also provides the method of any one of embodiments [1]-[25], wherein the pharmaceutical agent is an antineoplastic agent.

[27] The present invention also provides the method of any one of embodiments [1]-[26], wherein the pharmaceutical agent is a combination of two or more antineoplastic agents.

[28] The present invention also provides the method of any one of embodiments [1]-[27], wherein the pharmaceutical agent is an ionizable basic antineoplastic agent.

[29] The present invention also provides the method of any one of embodiments [1]-[28], wherein the pharmaceutical agent is an anthracycline chemotherapeutic agent, an anthracenedione, an amphiphilic drug, or a vinca alkaloid.

[30] The present invention also provides the method of embodiment [29], wherein the anthracycline chemotherapeutic agent is selected from the group of doxorubicin, epirubicin, and daunorubicin.

[31] The present invention also provides the method of embodiment [29], wherein the anthracenedione is mitoxantrone.

[32] The present invention also provides the method of embodiment [29], wherein the amphiphilic drug is a lipophilic amine.

[33] The present invention also provides the method of embodiment [20], wherein the vinca alkaloid is selected from the group of vincristine and vinblastine.

[34] The present invention also provides the method of any one of embodiments [1]-[28], wherein the pharmaceutical agent is an antineoplastic antibiotic.

[35] The present invention also provides the method of any one of embodiments [1]-[34], wherein the pharmaceutical agent is not camptothecin, or an analogue thereof.

[36] The present invention also provides the method of any one of embodiments [1]-[28], wherein the pharmaceutical agent is an alkylating agent.

[37] The present invention also provides the method of embodiment [36], wherein the alkylating agent is selected from the group of cyclophosphamide and mechlorethamine hydrochloride.

[38] The present invention also provides the method of any one of embodiments [1]-[28], wherein the pharmaceutical agent is a purine or pyrimidine derivative.

[39] The present invention also provides the method of embodiment [38], wherein the purine or pyrimidine derivative is 5-fluorouracil.

[40] The present invention also provides the method of any one of embodiments [1]-[39], wherein the temperature in step (a) is about 40° C. to about 70° C.

[41] The present invention also provides the method of any one of embodiments [1]-[40], wherein the temperature in step (a) is about 50° C. to about 60° C.

[42] The present invention also provides the method of any one of embodiments [1]-[41], wherein the solution is cooled in step (b) to a temperature of about 0° C. to about 30° C.

[43] The present invention also provides the method of any one of embodiments [1]-[42], wherein the solution in step (a) is prepared by the process comprising:

(i) contacting the liposomes and the aqueous solution of the acid;

(ii) homogenizing the solution; and

(iii) optionally removing any external acid.

[44] The present invention also provides the method of embodiment [43], wherein the external acid is removed in step (iii) by filtering the external acid.

[45] The present invention also provides the method of any one of embodiments [1]-[44], wherein the weak base is a membrane permeable amine.

[46] The present invention also provides the method of any one of embodiments [1]-[45], wherein the weak base is an ammonium salt or an alkyl amine.

[47] The present invention also provides the method of any one of embodiments [1]-[46], wherein the weak base is an ammonium salt having a mono- or multi-valent counterion.

[48] The present invention also provides the method of any one of embodiments [1]-[47], wherein the weak base is selected from the group of ammonium sulfate, ammonium hydroxide, ammonium acetate, ammonium chloride, ammonium phosphate, ammonium citrate, ammonium succinate, ammonium lactobionate, ammonium carbonate, ammonium tartarate, ammonium oxalate, and combinations thereof.

[49] The present invention also provides the method of any one of embodiments [1]-[47], wherein the weak base is alkyl-amine selected from the group of methyl amine, ethyl amine, diethyl amine, ethylene diamine, and propyl amine.

[50] The present invention also provides the method of any one of embodiments [1]-[49], further comprising, during or after step (c), removing any unloaded pharmaceutical agent.

[51] The present invention also provides the method of embodiment [50], wherein the removing of the unloaded drug employs removing the unloaded drug via cross filtration or dialysis.

[52] The present invention also provides the method of any one of embodiments [1]-[51], further comprising, after step (c), dehydrating the liposomes.

[53] The present invention also provides the method of embodiment [52], wherein the dehydrating is carried out at a pressure of below about 1 atm.

[54] The present invention also provides the method of embodiment [52], wherein the dehydrating is carried out with prior freezing of the liposomes.

[55] The present invention also provides the method of embodiment [52], wherein the dehydrating is carried out in the presence of one or more protective monosaccharide sugars, one or more protective disaccharide sugars, or a combination thereof.

[56] The present invention also provides the method of embodiment [55], wherein the protective sugar is selected from the group of trehalose, sucrose, maltose, and lactose.

[57] The present invention also provides the method of embodiment [52], further comprising rehydrating the liposomes after the dehydrating.

[58] The present invention also provides the method of any one of embodiments [1]-[57], wherein the liposomes are unilamellar vescicles.

[59] The present invention also provides the method of any one of embodiments [1]-[57], wherein the liposomes are multilamellar vescicles.

[60] The present invention also provides the method of any one of embodiments [1]-[59], wherein more than about 90 wt. % of the pharmaceutical agent is trapped in the liposomes.

[61] The present invention also provides the method of any one of embodiments [1]-[60], further comprising, after step (c), contacting the liposomes with a pharmaceutically acceptable carrier.

[62] The present invention also provides the method of any one of embodiments [1]-[61] wherein the acid is present in at least about 100 mM, at least about 200 mM, at least about 300 nM, at least about 400 nM, or at least about 500 nM.

[63] The present invention also provides a method for preparing a pharmaceutical composition comprising:

(a) contacting a solution of liposomes with a pharmaceutical agent in an aqueous solution of at least about 60 mM of an acid, at a temperature wherein the protonated form of the pharmaceutical agent is charged and is not capable of permeating the membrane of the liposomes, and wherein the unprotonated form of the pharmaceutical agent is uncharged and is capable of permeating the membrane of the liposomes;

(b) cooling the solution to a temperature at which the unprotonated form of the pharmaceutical agent is not capable of permeating the membrane of the liposomes;

(c) contacting the solution with a weak base, in an amount effective to raise the pH of the internal liposome to provide gradient loaded liposomes having a lower inside/higher outside pH gradient; and

(d) combining the liposomes with a pharmaceutically acceptable carrier to provide the pharmaceutical composition.

[64] The present invention also provides a method comprising administering the pharmaceutical composition of embodiment [63] to a mammal.

[65] The present invention also provides a method for treating a mammal inflicted with cancer, the method comprising administering the pharmaceutical composition of embodiment [63] to the mammal, wherein the pharmaceutical agent is an antineoplastic agent.

[66] The present invention also provides a method of embodiment [65], wherein the cancer is a tumor, ovarian cancer, small cell lung cancer (SCLC), non small cell lung cancer (NSCLC), leukemia, sarcoma, colorectal cancer, head cancer, neck cancer, or breast cancer.

[67] The present invention also provides a method of embodiment [65], wherein the administration of the antineoplastic agent, via the liposomal formulation, has a toxicity profile that is lower than the toxicity profile associated with the administration of the antineoplastic agent in the free form.

[68] The present invention also provides a method of embodiment [67], wherein the toxicity is selected from the group of gastrointestinal toxicity and cumulative dose-dependent irreversible cardiomyopathy.

[69] The present invention also provides a method of embodiment [65], wherein the administration of the antineoplastic agent has unpleasant side-effects that are lower in incidence, severity, or a combination thereof, than unpleasant side-effects associated with the administration of the antineoplastic agent in the free form.

[70] The present invention also provides a method of embodiment [69], wherein the unpleasant side-effects are selected from the group of myelosuppression, alopecia, mucositis, nausea, vomiting, and anorexia.

[71] A gradient loaded liposome having a lower inside/higher outside pH gradient, prepared by the process comprising:

(a) contacting a solution of liposomes with a pharmaceutical agent in an aqueous solution of at least about 60 mM of an acid, at a temperature wherein the protonated form of the pharmaceutical agent is charged and is not capable of permeating the membrane of the liposomes, and wherein the unprotonated form of the pharmaceutical agent is uncharged and is capable of permeating the membrane of the liposomes;

(b) cooling the solution to a temperature at which the unprotonated form of the pharmaceutical agent is not capable of permeating the membrane of the liposomes; and

(c) contacting the solution with a weak base, in an amount effective to raise the pH of the internal liposome to provide gradient loaded liposomes having a lower inside/higher outside pH gradient.

The following examples are given for purposes of illustration only and not by way of limitation on the scope of the invention.

The maximum tolerated dose for a formulation can be determined in an array of known animal models. For example, it can be determined using Test B.

Test Method B—Maximum Tolerated Dose (MTD)

Nude mice (NCr.nu/nu-mice) were administered each formulation by I.V. administration and the maximum tolerated dose (MTD) for each formulation was then determined. Typically a range of doses were given until an MTD was found, with 2 mice per dose group. Estimate of MTD was determined by evaluation of body weight, lethality, behavior changes, and/or signs at autopsy. Typical duration of the experiment is observation of the mice for four weeks, with body weight measurements twice per week.

The anti-cancer activity for a formulation can be determined in an array of known animal models. For example, it can be determined in rats using Test A.

Test Method A—Breast Cancer Xenograft Models

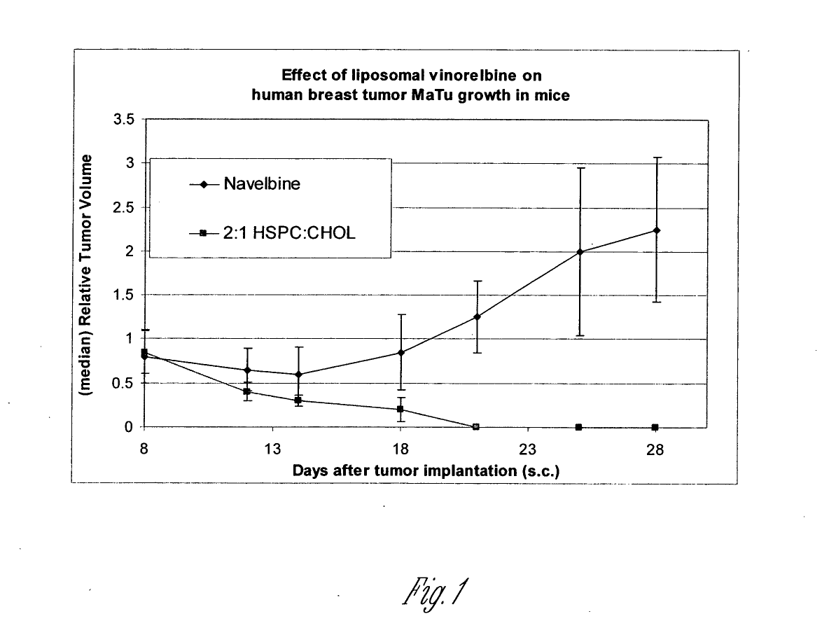

Nude mice were subcutaneously implanted with MaTu or MT-3 human breast carcinoma cells and were subsequently treated with formulations and a saline control. Treatment began on the tenth day after tumor implantation and consisted of dosing animals once or once a day for three consecutive days at the MTD of each respective agent. Tumor volumes were measured at several time points throughout the study with the study terminating about thirty-four days after tumor implantation. The median relative tumor volume (each individual tumor size measurement as related to the size of the tumor that was measured on day ten of the study) is plotted for each of the test articles. Representative data for a formulation comprising vinorelbine is shown in FIG. 1.

The invention is further defined by reference to the following examples. It will be apparent to those skilled in the art, that many modifications, both to materials and methods, may be practiced without departing from the purpose and interest of this invention.

### EXAMPLES

General Procedure for Liposome Preparation

Spray dried lipid powder containing various phospholipids including hydrogenated soy phosphatidyl choline (HSPC), cholesterol (Chol) and distearoylphosphatidylglycerol (DSPG) at various mole ratios were prepared. The studied lipid ratios were: HSPC:Chol:DSPG at a). 2:1:0 b). 2:1:0.1

Preparation of Spray Dried Lipid Powder

All lipid component were weighed out and were mixed in a round bottom flask, a chloroform : methanol 1:1 (v/v ) solvent was added to the lipid powder with a final lipid concentration around 200 mg/ml. The lipid solution was then spray dried to form lipid powder using a YAMATO GB-21 spray drier at a designed parameter setting. The residual solvent in the lipid powder was removed by drying under vacuum for three to five days.

Preparation of Drug Stock Solution

The requsite drug was weighed out and was dissolved in Water for Injection (WFI). The concentration of the drug stock solution is normally around 20 mg/ml. Stock solutions of Vinorelbine (NAV), Epirubicin (EPR), Mitoxantrone (MITO), Vincristine (VCR), and Doxorubicin (DOXO ) were prepared.

Preparation of Counter Ion Stock Solution

Based on pre-determined concentration, counter ion powder was weighed out and was dissolved in WFI. The final pH of the counter ion solution was adjusted to the designed pH if necessary. Solutions of the following counter ions were prepared: Citric Acid (CA), Ammonium Sulfate ((NH₄)₂SO₄), Tri-Ammonium Citrate ((NH₄)₃Citrate), and Lactobionic Acid (LBA).

Preparation of Pre-drug Loaded Liposome (Empty Liposome) by Probe Sonication from Either Lipid Film or Spray Dried Lipid Powder

Lipid film or lipid powder was weighed out and were hydrated with the desired counter ion solution at lipid concentration between 100 mg/ml to 150 mg/ml dependent on the experimental design. The hydrated solution was subjected to probe sonication until the solution became translucent. A typical temperature of sonication is 65° C. and a typical sonication time is 15 to 20 minutes. After completion of sonication, the liposomes were subjected to one of the following cleaning processes: a) Liposomes was cooled down to ambient temperature, a clear solution was applied to a sephadex G-50 column for buffer exchange with 9% sucrose; or b) upon completion of sonication, the liposomal solution was immediately diluted one to three with the same counter ion solution and that diluted solution was then subjected to ultrafiltration (U.F.) for cleaning/buffer exchange with 9% sucrose. The final lipid concentration of the liposome was kept around 50 mg/ml through the U.F. process.

Preparation of Liposomes by Homogenization from Spray Dried Lipid Powder

Lipid powder was weighed out and hydrated with the desired solution at lipid concentration between 50 mg/ml to 75 mg/ml. The hydrated solution was subjected to homogenization using a Niro homogenizer at 10,000 PSI at around 55° C. until the solution became translucent. A typical homogenization process took about 10 passes. After completion of homogenization, the liposomal solution was subjected to ultra filtration for cleaning/buffer exchange with 9% sucrose.

Preparation of Drug-loaded Liposome

A proper amount of empty liposome was measured, a calculated amount of drug stock solution was added to the empty liposome, the typical initial lipid to drug ratio by weight was 20 to 1. The system was then incubated at 55° C. and pH of the system was adjusted to the desired pH, typically is at pH 5.8 to pH 6.5 using sodium hydroxide. The system typically was given a loading/incubating time for 20 to 30 minutes. The post drug loaded liposome was then through either column separation or through U.F. processing buffer exchanged with 9% sucrose or with designed buffer (for quenching) [Note: allow for quenching in the wash phase—e.g. during column or U.F. cleanup or afterwards with the quenching agent spiked into the washed liposomes] and to remove any unloaded free drug.

The liposomes were filtered at ambient temperature through a cellulose acetate 0.22 micron filter.

### Example 4

### Liposomal Epirubicin

The epirubicin (EPR) stock solution was around 20 mg/ml. Lipid concentration of empty liposome was 50 mg/ml. A proper amount of empty liposome was measured, a calculated amount of drug stock solution was added to the empty liposome, and the lipid to drug ratio by weight was 20 to 1. The system was then incubated at 55° C. and pH of the system was adjusted to pH 6.0 using sodium hydroxide. The system was incubated at 55° C. for 20 minutes for drug loading. The post drug loaded liposome was then through cleaning process to remove any unloaded free drug by buffer exchange with 9% sucrose supplemented with the quenching agent. The liposomes were filtered at ambient temperature through a cellulose acetate 0.22 micron filter. Result of characterization of liposomes is shown in the Table below

| Lipid | Mole | Counter | | A750 | Size | | |
|---|---|---|---|---|---|---|---|
| Formulation | Ratio | Ion | Quencher | A600 | (nm) | Volume % | PH |
| HSPC/Chol | 2:1 | 150 mM | No | 0.346 | 53.3 | 100 | 5.62 |
| | | LBA | | | | | |
| HSPC/Chol | 2:1 | 50 mM | No | 1.073 | 78.8 | 80 | 7.01 |
| | | (NH₄)₃Citrate | | | | | |
| HSPC/Chol | 2:1 | 50 mM | No | 0.465 | 53.0 | 94 | 5.03 |
| | | CA | | | | | |
| HSPC/Chol | 2:1 | 75 mM | No | 0.876 | 61.5 | 89 | 6.68 |
| | | (NH₄)₂SO₄ | | | | | |
| HSPC/Chol | 2:1 | 75 mM | No | 0.926 | 74.0 | 72 | 6.12 |
| | | (NH₄)₂SO₄ | | | | | |
| HSPC/Chol | 2:1 | 50 mM | 9% Sucrose | 1.963 | 76.1 | 100 | 6.54 |
| | | CA | 10 mM NH₄Cl | | | | |
| HSPC/Chol | 2:1 | 50 mM | 9% Sucrose | 0.427 | 50.0 | 100 | 6.62 |
| | | CA | 10 mM NH₄Cl | | | | |
| HSPC/Chol | 2:1 | 200 mM | No | 0.302 | 47.4 | 100 | 6.78 |
| | | (NH₄)₂SO₄ | | | | | |
| HSPC/Chol/DSPG | 2:1:0.1 | 250 mM | 9% Sucrose | 0.753 | 43.4 | 100 | 6.38 |
| | | (NH₄)₂SO₄ | 2 mM NH₄Cl | | | | |
| HSPC/Chol/DSPG | 2:1:0.1 | 250 mM | 9% Sucrose | 1.083 | 41.6 | 100 | 6.32 |
| | | (NH₄)₂SO₄ | 2 mM NH₄Cl | | | | |

### Example 5

### Liposomal Doxorubicin

The doxorubicin stock solution was around 16 mg/ml. Lipid concentration of empty liposome was 40 mg/ml. A proper amount of empty liposome was measured, a calculated amount of drug stock solution was added to the empty liposome, and the lipid to drug ratio by weight was 20 to 1. The system was then incubated at 55° C. and pH of the system was adjusted to pH 7.4 using sodium hydroxide. The system was incubated at 55° C. for 20 minutes for drug loading. The post drug loaded liposome was then diluted one to three with quencher solution then through cleaning process to remove any unloaded free drug by buffer exchange with quencher solution. Upon completion of UF, the liposome were filtered through 0.2 μm PES filter. Result of characterization of liposomes is shown in Table below

| Lipid | Mole | Counter | | A750 | Size | | |
|---|---|---|---|---|---|---|---|
| Formulation | Ratio | Ion | Quencher | A600 | (nm) | Volume % | PH |
| HSPC/Chol/DSPG | 2:1:0.1 | 250 mM | 20 mM CaCl₂/ | 0.413 | 49.4 | 93 | 7.14 |
| | | (NH₄)₂SO₄ | 2 mM NH₄Cl | 0.952 | | | |

### Example 6

The following illustrate representative pharmaceutical dosage forms, containing a lipid-based dispersion of the invention, for therapeutic or prophylactic use in humans.

| | mg/ml |
|---|---|
| (i) Injection 1 (1 mg/ml) | |
| ‘Therapeutic Agent’ | 1.0 |
| Phosphatidyl choline | 40 |
| Cholesterol | 10 |
| Sucrose | 90 |
| 0.1 N Sodium hydroxide solution | q.s. |
| (pH adjustment to 7.0-7.5) | |
| Water for injection | q.s. ad 1 mL |
| (ii) Injection 2 (10 mg/ml) | |
| ‘Therapeutic Agent’ | 10 |
| Phosphatidyl choline | 60 |
| Cholesterol | 15 |
| Anionic Phospholipid | 3 |
| 0.1 N Sodium hydroxide solution | q.s. |
| (pH adjustment to 7.0-7.5) | |
| sucrose | 90 |
| Water for injection | q.s. ad 1 mL |

The above formulations may be obtained by conventional procedures well known in the pharmaceutical art.

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

It is appreciated that certain features of the invention, which are, for clarity, described in the context of separate embodiments, may also be provided in combination in a single embodiment. Conversely, various features of the invention which are for brevity, described in the context of a single embodiment, may also be provided separately or in any sub-combination.

1. A method of forming gradient loaded liposomes having a lower inside/higher outside pH gradient, the method comprising:
(a) contacting a solution of liposomes with a pharmaceutical agent in an aqueous solution of at least about 60 mM of an acid, at a temperature wherein the protonated form of the pharmaceutical agent is charged and is not capable of permeating the membrane of the liposomes, and wherein the unprotonated form of the pharmaceutical agent is uncharged and is capable of permeating the membrane of the liposomes;
(b) cooling the solution to a temperature at which the unprotonated form of the pharmaceutical agent is not capable of permeating the membrane of the liposomes; and
(c) contacting the solution with a weak base, in an amount effective to raise the pH of the internal liposome to provide gradient loaded liposomes having a lower inside/higher outside pH gradient.

2. The method of claim 1 wherein the liposomes comprise phosphatidylcholine.

3. The method of claim 1 wherein the liposomes comprise phosphatidylcholine selected from the group of distearoylphosphatidylcholine, hydrogenated soy phosphatidylcholine, hydrogenated egg phosphatidylcholine, dipalmitoylphosphatidylcholine, dimyristoylphosphatidylcholine, and dielaidoyl phosphatidyl chline.

4. The method of claim 1 wherein the liposomes further comprise cholesterol.

5. The method of claim 1 wherein the liposomes further comprise phosphatidylglycerol.

6. The method of claim 1 wherein the liposomes further comprise non-phosphatidyl lipids.

7. The method of claim 6 wherein the non-phosphatidyl lipids comprise sphingomyelin.

8. The method of claim 1 wherein the liposomes further comprise phosphatidylglycerol selected from the group of dimyristoylphosphatidylglycerol, dilaurylphosphatidylglycerol, dipalmitoylphosphatidylglycerol, and distearoylphosphatidylglycerol.

9. The method of claim 1 wherein the liposomes comprises phosphatidylcholine, and further comprises cholesterol.

10. The method of claim 1 wherein the liposomes comprises phosphatidylcholine, and further comprises cholesterol, wherein the molar ratio of the phosphatidylcholine to the cholesterol is about 1:0.01 to about 1:1.

11. The method of claim 1 wherein the liposomes comprises phosphatidylcholine, and further comprises cholesterol, wherein the molar ratio of the phosphatidylcholine to the cholesterol is about 1.5:1.0 to about 3.0:1.0.

12. The method of claim 1 wherein the liposomes are unilamellar and less than about 100 nm.

13. The method of claim 1 wherein the weight ratio of the liposomes to the pharmaceutical agent is up to about 200:1.

14. The method of claim 1 wherein the weight ratio of the liposomes to the pharmaceutical agent is about 1:1 to about 100:1.

15. The method of claim 1 wherein the weight ratio of the liposomes to the pharmaceutical agent is about 1:1 to about 50:1.

16. The method of claim 1 wherein the acid has an acid dissociation constant of less than about 1×10⁻².

17. The method of claim 1 wherein the acid has an acid dissociation constant of less than about 1×10⁻⁴.

18. The method of claim 1 wherein the acid has an acid dissociation constant of less than about 1×10⁻⁵.

19. The method of claim 1 wherein the acid has a permeability coefficient larger than about 1×10⁻⁴ cm/sec for the liposomes.

20. The method of claim 1 wherein the acid is selected from the group of formic acid, acetic acid, propanoic acid, butanoic acid, pentanoic acid, citric acid, oxalic acid, succinic acid, lactic acid, malic acid, tartaric acid, fumaric acid, benzoic acid, aconitic acid, veratric acid, phosphoric acid, sulfuric acid, and combinations thereof.

21. The method of claim 1 wherein the acid is citric acid.

22. The method in claim 1 wherein at least about 100 mM of an acid is employed.

23. The method of claim 1 wherein the pharmaceutical agent exists in a charged state when dissolved in an aqueous medium.

24. The method of claim 1 wherein the pharmaceutical agent is an organic compound that includes at least one acyclic or cyclic amino group, capable of being protonated.

25. The method of claim 1 wherein the pharmaceutical agent is an organic compound that includes at least one primary amine group, at least one secondary amine group, at least one tertiary amine group, at least one quaternary amine group, or any combination thereof.

26. The method of claim 1 wherein the pharmaceutical agent is an antineoplastic agent.

27. The method of claim 1 wherein the pharmaceutical agent is a combination of two or more antineoplastic agents.

28. The method of claim 1 wherein the pharmaceutical agent is an ionizable basic antineoplastic agent.

29. The method of claim 1 wherein the pharmaceutical agent is an anthracycline chemotherapeutic agent, an anthracenedione, an amphiphilic drug, or a vinca alkaloid.

30. The method of claim 29 wherein the anthracycline chemotherapeutic agent is selected from the group of doxorubicin, epirubicin, and daunorubicin.

31. The method of claim 29 wherein the anthracenedione is mitoxantrone.

32. The method of claim 29 wherein the amphiphilic drug is a lipophilic amine.

33. The method of claim 29 wherein the vinca alkaloid is selected from the group of vincristine and vinblastine.

34. The method of claim 1 wherein the pharmaceutical agent is an antineoplastic antibiotic.

35. The method of claim 1 wherein the pharmaceutical agent is not camptothecin, or an analogue thereof.

36. The method of claim 1 wherein the pharmaceutical agent is an alkylating agent.

37. The method of claim 36 wherein the alkylating agent is selected from the group of cyclophosphamide and mechlorethamine hydrochloride.

38. The method of claim 1 wherein the pharmaceutical agent is a purine or pyrimidine derivative.

39. The method of claim 38 wherein the purine or pyrimidine derivative is 5-fluorouracil.

40. The method of claim 1 wherein the temperature in step (a) is about 40° C. to about 70° C.

41. The method of claim 1 wherein the temperature in step (a) is about 50° C. to about 60° C.

42. The method of claim 1 wherein the solution is cooled in step (b) to a temperature of about 0° C. to about 30° C.

43. The method of claim 1 wherein the solution in step (a) is prepared by the process comprising:
(i) contacting the liposomes and the aqueous solution of the acid;
(ii) homogenizing the solution; and
(iii) optionally removing any external acid.

44. The method of claim 43 wherein the external acid is removed in step (iii) by filtering the external acid.

45. The method of claim 1 wherein the weak base is a membrane permeable amine.

46. The method of claim 1 wherein the weak base is an ammonium salt or an alkyl amine.

47. The method of claim 1 wherein the weak base is an ammonium salt having a mono- or multi-valent counterion.

48. The method of claim 1 wherein the weak base is selected from the group of ammonium sulfate, ammonium hydroxide, ammonium acetate, ammonium chloride, ammonium phosphate, ammonium citrate, ammonium succinate, ammonium lactobionate, ammonium carbonate, ammonium tartarate, ammonium oxalate, and combinations thereof.

49. The method of claim 1 wherein the weak base is alkyl-amine selected from the group of methyl amine, ethyl amine, diethyl amine, ethylene diamine, and propyl amine.

50. The method of claim 1 further comprising, during or after step (c), removing any unloaded pharmaceutical agent.

51. The method of claim 50 wherein the removing of the unloaded drug employs removing the unloaded drug via cross filtration or dialysis.

52. The method of claim 1 further comprising, after step (c), dehydrating the liposomes.

53. The method of claim 52 wherein the dehydrating is carried out at a pressure of below about 1 atm.

54. The method of claim 52 wherein the dehydrating is carried out with prior freezing of the liposomes.

55. The method of claim 52 wherein the dehydrating is carried out in the presence of one or more protective monosaccharide sugars, one or more protective disaccharide sugars, or a combination thereof.

56. The method of claim 55 wherein the protective sugar is selected from the group of trehalose, sucrose, maltose, and lactose.

57. The method of claim 52 further comprising rehydrating the liposomes after the dehydrating.

58. The method of claim 1 wherein the liposomes are unilamellar vescicles.

59. The method of claim 1 wherein the liposomes are multilamellar vescicles.

60. The method of claim 1 wherein more than about 90 wt. % of the pharmaceutical agent is trapped in the liposomes.

61. The method of claim 1 further comprising, after step (c), contacting the liposomes with a pharmaceutically acceptable carrier.

62. The method of claim 1 wherein the acid is present in at least about 300 nM.

63. A method for preparing a pharmaceutical composition comprising:
(a) contacting a solution of liposomes with a pharmaceutical agent in an aqueous solution of at least about 60 mM of an acid, at a temperature wherein the protonated form of the pharmaceutical agent is charged and is not capable of permeating the membrane of the liposomes, and wherein the unprotonated form of the pharmaceutical agent is uncharged and is capable of permeating the membrane of the liposomes;
(b) cooling the solution to a temperature at which the unprotonated form of the pharmaceutical agent is not capable of permeating the membrane of the liposomes;
(c) contacting the solution with a weak base, in an amount effective to raise the pH of the internal liposome to provide gradient loaded liposomes having a lower inside/higher outside pH gradient; and
(d) combining the liposomes with a pharmaceutically acceptable carrier to provide the pharmaceutical composition.

64. A method comprising administering the pharmaceutical composition of claim 63 to a mammal.

65. A method for treating a mammal inflicted with cancer, the method comprising administering the pharmaceutical composition of claim 63 to the mammal, wherein the pharmaceutical agent is an antineoplastic agent.

66. The method of claim 65 wherein the cancer is a tumor, ovarian cancer, small cell lung cancer (SCLC), non small cell lung cancer (NSCLC), leukemia, sarcoma, colorectal cancer, head cancer, neck cancer, or breast cancer.

67. The method of claim 65 wherein the administration of the antineoplastic agent, via the liposomal formulation, has a toxicity profile that is lower than the toxicity profile associated with the administration of the antineoplastic agent in the free form.

68. The method of claim 67 wherein the toxicity is selected from the group of gastrointestinal toxicity and cumulative dose-dependent irreversible cardiomyopathy.

69. The method of claim 65 wherein the administration of the antineoplastic agent has unpleasant side-effects that are lower in incidence, severity, or a combination thereof, than unpleasant side-effects associated with the administration of the antineoplastic agent in the free form.

70. The method of claim 69 wherein the unpleasant side-effects are selected from the group of myelosuppression, alopecia, mucositis, nausea, vomiting, and anorexia.

71. A gradient loaded liposome having a lower inside/higher outside pH gradient prepared by the process comprising:
(a) contacting a solution of liposomes with a pharmaceutical agent in an aqueous solution of at least about 60 mM of an acid, at a temperature wherein the protonated form of the pharmaceutical agent is charged and is not capable of permeating the membrane of the liposomes, and wherein the unprotonated form of the pharmaceutical agent is uncharged and is capable of permeating the membrane of the liposomes;
(b) cooling the solution to a temperature at which the unprotonated form of the pharmaceutical agent is not capable of permeating the membrane of the liposomes; and
(c) contacting the solution with a weak base, in an amount effective to raise the pH of the internal liposome to provide gradient loaded liposomes having a lower inside/higher outside pH gradient.
